(19) United States
(12) Patent Application Publication
(10) Pub. No.: US 2002/0081610 A1
(43) Pub. Date: Jun. 27, 2002

(54) Assays and materials for embryonic gene expression

(75) Inventors: Ali Hemmati-Brivanlou, New York, NY (US); Curtis R. Altmann, New York, NY (US)

Correspondence Address: DARBY & DARBY P.C., 805 Third Avenue, New York, NY, 10022, US

(73) Assignee: The Rockefeller University

(21) Appl. No.: 09/910,943

(22) Filed: Jul. 23, 2001

Related U.S. Application Data
(60) Provisional application No. 60/219,658, filed on Jul. 21, 2000.

(51) Int. Cl. C12Q 1/68; C07H 21/04; C12M 1/34

(52) U.S. Cl. 435/6; 435/287.2; 536/24.3

(57) ABSTRACT

The present invention relates to methods for detecting differential expression of embryonic gene products known to play a fundamental role in the embryonic developmental process using nucleic acid arrays containing Xenopus embryonic gene sequences as set forth in Appendix 1. This allows the detection of the expression of differentially expressed genes in embryonic cells, for diagnosing developmental disorders or identifying different types of embryonic cells.

This application claims priority under 35 U.S.C. § 119(e) to U.S. Provisional patent application serial No. 60/219,658 filed on Jul. 21, 2000. The contents of the priority application are incorporated herein, by reference, in their entirety.

This invention was made with government support under Grant No. ______ awarded by ______. The United States government may have certain rights to this invention pursuant to the terms of that grant.

### FIELD OF THE INVENTION

The present invention relates to genes that are differentially expressed in developing embryos as well as to their gene products. Accordingly, the genes and gene products of this invention are useful for the treatment of various disease and disorders associated with abnormal embryonic development. The invention also relates to microarrays that have probes for one or more of these differentially expressed genes. Such microarrays are useful for detecting expression of these differentially expressed genes in cells, for diagnosing developmental disorders that involve aberrant or abnormal expression of one or more of these genes, for “fingerprinting” or identifying different types of embryonic cells, and for determining the function of an unknown gene or gene product based on the expression profile it induces.

### BACKGROUND OF THE INVENTION

The development of high throughput approaches in molecular biology, where a large number of genes can be analyzed simultaneously, has provided researchers with the unique opportunity to look at biological responses globally as opposed to one gene or one pathway at a time (Schena, Bioessays 1996, 18:427; see also Schena et al., Proc. Natl. Acad. Sci. USA 1996, 93:10614). This approach complements genetic approaches (when available), and allows genome wide analysis to be applied to non-genetic systems. A great deal of effort and interest has been spent applying these technologies to human and mouse models as well as invertebrate systems, but this type of approach has not been applied to a vertebrate developmental model system.

During embryonic development, signals from one group of cells influence cell fate decisions of other cells in a process known as induction. These inductive signals act within the embryo both in the context of time and space to induce differentiation of various cell types. Differentiation of cells is the result of stable changes in gene expression (which in most circumstances is not reversible) and the expression of cell type specific genes. Current methods for analyzing induction and differentiation rely on reverse transcription of the mRNA message and polymerase chain reaction (RT-PCR) using primers which amplify previously defined cell type specific genes or “markers (such as NCAM for neural fates and keratin for epidermal fates). There are approximately 200 cell type specific molecular markers reported and employed by various laboratories to study embryonic induction. While extremely sensitive and useful, the number of markers that can be assessed in a single experiment is limited to about 20 and requires the researcher to make a subjective selection of markers for a given assay. This approach works well when examining the formation of particular tissue type but is limited when one is assaying a gene of unknown function. Thus there is a need in the art for a more robust approach to functional genomics of embryogenesis.

### SUMMARY OF THE INVENTION

The present invention provides a nucleic acid array containing a single nucleic acid species of a Xenopus embryonic gene product set forth in Appendix 1. In addition, the invention provides an isolated nucleic acid comprising a sequence corresponding to or complementary to a sequence of not less than 20, preferably not less than 50, and more preferably not less than 100, contiguous nucleotides of any one of the sequences of Appendix 1. These sequences correspond to the gene products listed in the tables of Appendix 2, as can readily be determined by one of ordinary skill from the sequence information.

The invention further provides a method for detecting differential expression of embryonic genes by contacting a nucleic acid array having one or more genes expressed in embryonic cells but not mature cells with a sample and a nucleic acid preparation and detecting differential hybridization of nucleic acids from the sample cells compared to the control cells.

The invention further provides a method for detecting defects in embryonic development using a nucleic acid array of Xenopus gene products known to play a fundamental role in the development process and also detecting the difference in expression of a fundamental gene in sample cells relative to the standard, indicative of a developmental defect.

### BRIEF DESCRIPTION OF THE DRAWINGS

FIG. 1 is a scatter plot comparing expression levels of genes in pre-MBT Xenopus embryos (i.e., stage 6 embryos; horizontal axis) and early gastrula embryos (vertical axis), as determined by two-color fluorescence hybridization to microarrays having probes for the Xenopus clones described herein. Points along the diagonal (•) identify genes expressed at levels within a factor of two in both types of embryos. Points below and to the right of the diagonal (×) identify genes that are expressed at higher levels (i.e., by a factor of two or more) in pre-MBT embryos, whereas the points above and to the left of the diagonal (+) identify genes that are expressed at higher levels in early gastrula stage embryos.

FIG. 2 shows, by in situ hybridization, the spatially restricted expression of certain exemplary genes (contained within the clones described herein) in Xenopus embryos at different stages of development.

FIG. 2A, and the insert beneath, show expression of the gene S10-8-B8 in a gastrula stage embryo;

FIGS. 2B and 2C show expression of the gene S10-8-B8 in a neutrula stage embryo;

FIG. 2D shows expression of the gene S10-3-C9 in a gastrula stage embryo; and

FIG. 2E shows expression of the gene S10-3-C9 in a neurula stage embryo.

### DETAILED DESCRIPTION OF THE INVENTION

The present invention provides gene expression chips based on genes found in frog embryos during development. Over 1,000 genes were included in the first chips, of which nearly 200 unique sequences (out of about 900 sequences obtained) were found. These gene products, sequences thereof, and nucleic acid arrays containing them form the basis for this invention.

In order to apply high throughput approaches to a developmental model system, a robotic device was built for preparing DNA microarrays (Brown and Botstein Nat. Genet., 1999, 21 (1 Suppl):33) and a prototype Xenopus laevis microarray prepared. Xenopus embryos are an ideal system for the study of early vertebrate development because of the abundance of biological material (up to 10,000 embryos/day/female), and embryonic development can be followed from fertilization onward.

The application of microarrays to vertebrate development would allow important biological and medically relevant questions to be addressed. Microarrays can be used to determine changes in gene expression with respect to time, gene expression changes in the context of space, gene expression changes in tissue explants in response to added protein and gene expression changes in tissue explants in response to expressed mRNA. Additional applications include the use of microarrays for “fingerprinting” of cell types. The power of micrarrays to identify subtle differences in cell types has been recently highlighted for the diagnosis of B-cell lymphomas subtypes. Xenopus laevis offers advantages for the study of the molecular basis of embryonic cell fate decisions, as the cells are pluripotent and since the source of nutrient is internal, these cells can be cultured in vitro without the need of extrinsic factors. Thus, the effects of individual activities can be assessed without the influences derived from the growth media.

The use of a microarray based approach provides a significant improvement over previous methods because provides an objective method for examining gene function globally and represents an obvious choice of using information generated by the different ongoing EST projects.

Initial work done with frogs is significant for murine, and human, embryogenesis. This initial work provides corresponding human embryonic chips useful in monitoring in vitro fertilization and as a means for evaluating fetal cells obtained by amniocentesis. The invention provides methods of detecting particular genetic phenomena in embryos using such chips. The close evolutionary relationships of signaling molecules means that information derived from the frog embryos are relevant to human gene expression.

In addition to a “gene chip” that incorporates the genes, the invention further provides a method for detecting differential gene expression, particularly of Xenopus genes, but also an ortholog of any such gene from another species, e.g., human.

As used herein, the term “nucleic acid array” refers to “gene chips” and related arrays of oligonucleotides, cDNAs, and other nucleic acids, which are well known in the art (see for example the following: U.S. Pat. Nos. 6,045,996; 6,040,138; 6,027,880; 6,020,135; 5,968,740; 5,959,098; 5,945,334; 5,885,837; 5,874,219; 5,861,242; 5,843,655; 5,837,832; 5,677,195 and 5,593,839).

Although as exemplified, the lessons of the present invention are learned from embryonic genes expressed during Xenopus differentiation and development, they apply equally to other animal developmental systems. In particular, the gene embryonic gene arrays of the present invention provide a robust and powerful system for evaluating developmental processes in mammals, including mice, and in particular in humans.

### General Definitions

As used herein, the term “embryonic gene product refers to a gene product expressed during embryogenesis. Preferably such a gene product is not expressed in mature cells. Thus, the gene product represents a specific embryogenic gene. Such genes are likely involved in developmental processes. Differential expression of these genes is critical to appropriate development, and differentiation with time and location of cells in a developing organism.

In a specific embodiment, the term “about” or “approximately” means within 20%, preferably within 10%, and more preferably within 5% of a given value or range. Alternatively, the term can mean within an acceptable error range given the particular type of data or the nature of the quantity for which a value is provided. In biological systems, frequently an order of magnitude variance is tolerable; preferably the variance is around 2-fold.

As used herein, the term “isolated” means that the referenced material is free of components found in the natural environment in which the material is normally found. In particular, isolated biological material is free of cellular components. In the case of nucleic acid molecules, an isolated nucleic acid includes a PCR product, an isolated mRNA, a cDNA, or a restriction fragment. In another embodiment, an isolated nucleic acid is preferably excised from the chromosome in which it may be found, and more preferably is no longer joined to non-regulatory, non-coding regions, or to other genes, located upstream or downstream of the gene contained by the isolated nucleic acid molecule when found in the chromosome. In yet another embodiment, the isolated nucleic acid lacks one or more introns. Isolated nucleic acid molecules can be inserted into plasmids, cosmids, artificial chromosomes, and the like. Thus, in a specific embodiment, a recombinant nucleic acid is an isolated nucleic acid. An isolated protein may be associated with other proteins or nucleic acids, or both, with which it associates in the cell, or with cellular membranes if it is a membrane-associated protein. An isolated organelle, cell, or tissue is removed from the anatomical site in which it is found in an organism. An isolated material may be, but need not be, purified.

The term “purified” as used herein refers to material that has been isolated under conditions that reduce or eliminate unrelated materials, i.e., contaminants. For example, a purified protein is preferably substantially free of other proteins or nucleic acids with which it is associated in a cell; a purified nucleic acid molecule is preferably substantially free of proteins or other unrelated nucleic acid molecules with which it can be found within a cell. As used herein, the term “substantially free” is used operationally, in the context of analytical testing of the material. Preferably, purified material substantially free of contaminants is at least 50% pure; more preferably, at least 90% pure, and more preferably still at least 99% pure. Purity can be evaluated by chromatography, gel electrophoresis, immunoassay, composition analysis, biological assay, and other methods known in the art.

### Molecular Biology Definitions

In accordance with the present invention there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization [B. D. Hames & S. J. Higgins eds. (1985)]; Transcription And Translation [B. D. Hames & S. J. Higgins, eds. (1984)]; Animal Cell Culture [R. I. Freshney, ed. (1986)]; Immobilized Cells And Enzymes [IRL Press, (1986)]; B. Perbal, A Practical Guide To Molecular Cloning (1984); F. M. Ausubel et al. (eds.), Current Protocols in Molecular Biology, John Wiley & Sons, Inc. (1994).

“Amplification” of DNA as used herein denotes the use of polymerase chain reaction (PCR) to increase the concentration of a particular DNA sequence within a mixture of DNA sequences. For a description of PCR see Saiki et al., Science, 239:487, 1988.

“Chemical sequencing” of DNA denotes methods such as that of Maxam and Gilbert (Maxam-Gilbert sequencing, Maxam and Gilbert, Proc. Natl. Acad. Sci. USA, 74:560, 1977), in which DNA is randomly cleaved using individual base-specific reactions.

“Enzymatic sequencing” of DNA denotes methods such as that of Sanger (Sanger et al., 1977, Proc. Natl. Acad. Sci. USA, 74:5463, 1977), in which a single-stranded DNA is copied and randomly terminated using DNA polymerase, including variations thereof well-known in the art.

As used herein, “sequence-specific oligonucleotides” refers to related sets of oligonucleotides that can be used to detect allelic variations or mutations in the gene.

A “nucleic acid molecule” refers to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogs thereof, such as phosphorothioates and thioesters, in either single stranded form, or a double-stranded helix. Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear (e.g., restriction fragments) or circular DNA molecules, plasmids, and chromosomes. In discussing the structure of particular double-stranded DNA molecules, sequences may be described herein according to the normal convention of giving only the sequence in the 5′ to 3′ direction along the nontranscribed strand of DNA (i.e., the strand having a sequence homologous to the mRNA). A “recombinant DNA molecule” is a DNA molecule that has undergone a molecular biological manipulation.

A “polynucleotide” or “nucleotide sequence” is a series of nucleotide bases (also called “nucleotides”) in DNA and RNA, and means any chain of two or more nucleotides. A nucleotide sequence typically carries genetic information, including the information used by cellular machinery to make proteins and enzymes. These terms include double or single stranded genomic and cDNA, RNA, any synthetic and genetically manipulated polynucleotide, and both sense and anti-sense polynucleotide (although only sense stands are being represented herein). This includes single- and double-stranded molecules, i.e., DNA-DNA, DNA-RNA and RNA-RNA hybrids, as well as “protein nucleic acids” (PNA) formed by conjugating bases to an amino acid backbone. This also includes nucleic acids containing modified bases, for example thio-uracil, thio-guanine and fluoro-uracil.

The polynucleotides herein may be flanked by natural regulatory (expression control) sequences, or may be associated with heterologous sequences, including promoters, internal ribosome entry sites (IRES) and other ribosome binding site sequences, enhancers, response elements, suppressors, signal sequences, polyadenylation sequences, introns, 5′- and 3′- non-coding regions, and the like. The nucleic acids may also be modified by many means known in the art. Non-limiting examples of such modifications include methylation, “caps”, substitution of one or more of the naturally occurring nucleotides with an analog, and internucleotide modifications such as, for example, those with uncharged linkages (e.g., methyl phosphonates, phosphotriesters, phosphoroamidates, carbamates, etc.) and with charged linkages (e.g., phosphorothioates, phosphorodithioates, etc.). Polynucleotides may contain one or more additional covalently linked moieties, such as, for example, proteins (e.g., nucleases, toxins, antibodies, signal peptides, poly-L-lysine, etc.), intercalators (e.g., acridine, psoralen, etc.), chelators (e.g., metals, radioactive metals, iron, oxidative metals, etc.), and alkylators. The polynucleotides may be derivatized by formation of a methyl or ethyl phosphotriester or an alkyl phosphoramidate linkage. Furthermore, the polynucleotides herein may also be modified with a label capable of providing a detectable signal, either directly or indirectly. Exemplary labels include radioisotopes, fluorescent molecules, biotin, and the like.

The term “host cell” means any cell of any organism that is selected, modified, transformed, grown, or used or manipulated in any way, for the production of a substance by the cell, for example the expression by the cell of a gene, a DNA or RNA sequence, a protein or an enzyme. Host cells can further be used for screening or other assays, as described infra.

Proteins and enzymes are made in the host cell using instructions in DNA and RNA, according to the genetic code. Generally, a DNA sequence having instructions for a particular protein or enzyme is “transcribed” into a corresponding sequence of RNA. The RNA sequence in turn is “translated” into the sequence of amino acids which form the protein or enzyme. An “amino acid sequence” is any chain of two or more amino acids. Each amino acid is represented in DNA or RNA by one or more triplets of nucleotides. Each triplet forms a codon, corresponding to an amino acid. For example, the amino acid lysine (Lys) can be coded by the nucleotide triplet or codon AAA or by the codon AAG. (The genetic code has some redundancy, also called degeneracy, meaning that most amino acids have more than one corresponding codon.) Because the nucleotides in DNA and RNA sequences are read in groups of three for protein production, it is important to begin reading the sequence at the correct amino acid, so that the correct triplets are read. The way that a nucleotide sequence is grouped into codons is called the “reading frame.”

A “coding sequence” or a sequence “encoding” an expression product, such as a RNA, polypeptide, protein, or enzyme, is a nucleotide sequence that, when expressed, results in the production of that RNA, polypeptide, protein, or enzyme, i.e., the nucleotide sequence encodes an amino acid sequence for that polypeptide, protein or enzyme. A coding sequence for a protein may include a start codon (usually ATG) and a stop codon.

The term “gene”, also called a “structural gene” means a DNA sequence that codes for or corresponds to a particular sequence of amino acids which comprise all or part of one or more proteins or enzymes, and may or may not include regulatory DNA sequences, such as promoter sequences, which determine for example the conditions under which the gene is expressed. Some genes, which are not structural genes, may be transcribed from DNA to RNA, but are not translated into an amino acid sequence. Other genes may function as regulators of structural genes or as regulators of DNA transcription.

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined for example, by mapping with nuclease S1), as well as protein binding domains (consensus sequences) responsible for the binding of RNA polymerase.

A coding sequence is “under the control” or “operatively associated with” of transcriptional and translational control sequences in a cell when RNA polymerase transcribes the coding sequence into mRNA, which is then trans-RNA spliced (if it contains introns) and translated into the protein encoded by the coding sequence.

The terms “express” and “expression” mean allowing or causing the information in a gene or DNA sequence to become manifest, for example producing a protein by activating the cellular functions involved in transcription and translation of a corresponding gene or DNA sequence. A DNA sequence is expressed in or by a cell to form an “expression product” such as a protein. The expression product itself, e.g. the resulting protein, may also be said to be “expressed” by the cell. An expression product can be characterized as intracellular, extracellular or secreted. The term “intracellular” means something that is inside a cell. The term “extracellular” means something that is outside a cell. A substance is “secreted” by a cell if it appears in significant measure outside the cell, from somewhere on or inside the cell.

The term “transfection” means the introduction of a foreign nucleic acid into a cell. The term “transformation” means the introduction of a “foreign” (i.e. extrinsic or extracellular) gene, DNA or RNA sequence to a host cell, so that the host cell will express the introduced gene or sequence to produce a desired substance, typically a protein or enzyme coded by the introduced gene or sequence. The introduced gene or sequence may also be called a “cloned” or “foreign” gene or sequence, may include regulatory or control sequences, such as start, stop, promoter, signal, secretion, or other sequences used by a cell's genetic machinery. The gene or sequence may include nonfunctional sequences or sequences with no known function. A host cell that receives and expresses introduced DNA or RNA has been “transformed” and is a “transformant” or a “clone.” The DNA or RNA introduced to a host cell can come from any source, including cells of the same genus or species as the host cell, or cells of a different genus or species.

The terms “vector”, “cloning vector” and “expression vector” mean the vehicle by which a DNA or RNA sequence (e.g. a foreign gene) can be introduced into a host cell, so as to transform the host and promote expression (e.g. transcription and translation) of the introduced sequence. Vectors include plasmids, phages, viruses, etc.; they are discussed in greater detail below.

Vectors typically comprise the DNA of a transmissible agent, into which foreign DNA is inserted. A common way to insert one segment of DNA into another segment of DNA involves the use of enzymes called restriction enzymes that cleave DNA at specific sites (specific groups of nucleotides) called restriction sites. A “cassette” refers to a DNA coding sequence or segment of DNA that codes for an expression product that can be inserted into a vector at defined restriction sites. The cassette restriction sites are designed to ensure insertion of the cassette in the proper reading frame. Generally, foreign DNA is inserted at one or more restriction sites of the vector DNA, and then is carried by the vector into a host cell along with the transmissible vector DNA. A segment or sequence of DNA having inserted or added DNA, such as an expression vector, can also be called a “DNA construct.” A common type of vector is a “plasmid”, which generally is a self-contained molecule of double-stranded DNA, usually of bacterial origin, that can readily accept additional (foreign) DNA and which can readily introduced into a suitable host cell. A plasmid vector often contains coding DNA and promoter DNA and has one or more restriction sites suitable for inserting foreign DNA. Coding DNA is a DNA sequence that encodes a particular amino acid sequence for a particular protein or enzyme. Promoter DNA is a DNA sequence which initiates, regulates, or otherwise mediates or controls the expression of the coding DNA. Promoter DNA and coding DNA may be from the same gene or from different genes, and may be from the same or different organisms. A large number of vectors, including plasmid and fungal vectors, have been described for replication and/or expression in a variety of eukaryotic and prokaryotic hosts. Non-limiting examples include pKK plasmids (Clonetech), pUC plasmids, pET plasmids (Novagen, Inc., Madison, Wis.), pRSET or pREP plasmids (Invitrogen, San Diego, Calif.), or pMAL plasmids (New England Biolabs, Beverly, Mass.), and many appropriate host cells, using methods disclosed or cited herein or otherwise known to those skilled in the relevant art. Recombinant cloning vectors will often include one or more replication systems for cloning or expression, one or more markers for selection in the host, e.g. antibiotic resistance, and one or more expression cassettes.

The term “expression system” means a host cell and compatible vector under suitable conditions, e.g. for the expression of a protein coded for by foreign DNA carried by the vector and introduced to the host cell. Common expression systems include E. coli host cells and plasmid vectors, insect host cells and Baculovirus vectors, and mammalian host cells and vectors. In a specific embodiment, the protein of interest is expressed in Xenopus oocytes or embryonic cells.

The term “heterologous” refers to a combination of elements not naturally occurring. For example, heterologous DNA refers to DNA not naturally located in the cell, or in a chromosomal site of the cell. Preferably, the heterologous DNA includes a gene foreign to the cell. A heterologous expression regulatory element is a such an element operatively associated with a different gene than the one it is operatively associated with in nature. In the context of the present invention, a gene encoding a protein of interest is heterologous to the vector DNA in which it is inserted for cloning or expression, and it is heterologous to a host cell containing such a vector, in which it is expressed, e.g., a Xenopus oocyte.

The terms “mutant” and “mutation” mean any detectable change in genetic material, e.g. DNA, or any process, mechanism, or result of such a change. This includes gene mutations, in which the structure (e.g. DNA sequence) of a gene is altered, any gene or DNA arising from any mutation process, and any expression product (e.g. protein or enzyme) expressed by a modified gene or DNA sequence. The term “variant” may also be used to indicate a modified or altered gene, DNA sequence, enzyme, cell, etc., i.e., any kind of mutant. The present invention includes mutants and variants of the sequence of Appendix 1, which are the gene products listed in Appendix 2.

“Sequence-conservative variants” of a polynucleotide sequence are those in which a change of one or more nucleotides in a given codon position results in no alteration in the amino acid encoded at that position. The invention includes sequence-conservative variants of the sequences of Appendix 1, which are the gene products listed in Appendix 2.

“Function-conservative variants” are those in which a given amino acid residue in a protein or enzyme has been changed without altering the overall conformation and function of the polypeptide, including, but not limited to, replacement of an amino acid with one having similar properties (such as, for example, polarity, hydrogen bonding potential, acidic, basic, hydrophobic, aromatic, and the like). Amino acids with similar properties are well known in the art. For example, arginine, histidine and lysine are hydrophilic-basic amino acids and may be interchangeable. Similarly, isoleucine, a hydrophobic amino acid, may be replaced with leucine, methionine or valine. Such changes are expected to have little or no effect on the apparent molecular weight or isoelectric point of the protein or polypeptide. Amino acids other than those indicated as conserved may differ in a protein or enzyme so that the percent protein or amino acid sequence similarity between any two proteins of similar function may vary and may be, for example, from 70% to 99% as determined according to an alignment scheme such as by the Cluster Method, wherein similarity is based on the MEGALIGN algorithm. A “function-conservative variant” also includes a polypeptide or enzyme which has at least 60% amino acid identity as determined by BLAST or FASTA algorithms, preferably at least 75%, most preferably at least 85%, and even more preferably at least 90%, and which has the same or substantially similar properties or functions as the native or parent protein or enzyme to which it is compared. The invention includes sequence-conservative variants of the sequences of Appendix 1, which are the gene products listed in Appendix 2.

As used herein, the term “homologous” in all its grammatical forms and spelling variations refers to the relationship between proteins that possess a “common evolutionary origin,” including proteins from superfamilies (e.g., the immunoglobulin superfamily) and homologous proteins from different species (e.g., myosin light chain, etc.) (Reeck et al., Cell 50:667, 1987). Such proteins (and their encoding genes) have sequence homology, as reflected by their sequence similarity, whether in terms of percent similarity or the presence of specific residues or motifs at conserved positions. The invention includes one or more homologous coding sequences to those set forth in Appendix 1, which are the gene products listed in Appendix 2, particularly homologs from other species (orthologs), such as humans.

Accordingly, the term “sequence similarity” in all its grammatical forms refers to the degree of identity or correspondence between nucleic acid or amino acid sequences of proteins that may or may not share a common evolutionary origin (see Reeck et al., supra). However, in common usage and in the instant application, the term “homologous,” when modified with an adverb such as “highly,” may refer to sequence similarity and may or may not relate to a common evolutionary origin.

In a specific embodiment, two DNA sequences are “substantially homologous” or “substantially similar” when at least about 80%, and most preferably at least about 90 or 95% of the nucleotides match over the defined length of the DNA sequences, as determined by sequence comparison algorithms, such as BLAST, FASTA, DNA Strider, etc. An example of such a sequence is an allelic or species variant of the specific genes of the invention. Sequences that are substantially homologous can be identified by comparing the sequences using standard software available in sequence data banks, or in a Southern hybridization experiment under, for example, stringent conditions as defined for that particular system.

Similarly, in a particular embodiment, two amino acid sequences are “substantially homologous” or “substantially similar” when greater than 80% of the amino acids are identical, or greater than about 90% are similar (functionally identical). Preferably, the similar or homologous sequences are identified by alignment using, for example, the GCG (Genetics Computer Group, Program Manual for the GCG Package, Version 7, Madison, Wis.) pileup program, or any of the programs described above (BLAST, FASTA, etc.).

A nucleic acid molecule is “hybridizable” to another nucleic acid molecule, such as a cDNA, genomic DNA, or RNA, when a single stranded form of the nucleic acid molecule can anneal to the other nucleic acid molecule under the appropriate conditions of temperature and solution ionic strength (see Sambrook et al., supra). The conditions of temperature and ionic strength determine the “stringency” of the hybridization. For preliminary screening for homologous nucleic acids, low stringency hybridization conditions, corresponding to a Tₘ (melting temperature) of 55° C., can be used, e.g., 5×SSC, 0.1% SDS, 0.25% milk, and no formamide; or 30% formamide, 5×SSC, 0.5% SDS). Moderate stringency hybridization conditions correspond to a higher Tₘ, e.g., 40% formamide, with 5× or 6×SCC. High stringency hybridization conditions correspond to the highest Tₘ, e.g., 50% formamide, 5× or 6×SCC. SCC is a 0.15M NaCl, 0.015M Na-citrate. Hybridization requires that the two nucleic acids contain complementary sequences, although depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the greater the value of Tₘ for hybrids of nucleic acids having those sequences. The relative stability (corresponding to higher Tₘ) of nucleic acid hybridizations decreases in the following order: RNA:RNA, DNA:RNA, DNA:DNA. For hybrids of greater than 100 nucleotides in length, equations for calculating Tₘ have been derived (see Sambrook et al., supra, 9.50-9.51). For hybridization with shorter nucleic acids, i.e., oligonucleotides, the position of mismatches becomes more important, and the length of the oligonucleotide determines its specificity (see Sambrook et al., supra, 11.7-11.8). A minimum length for a hybridizable nucleic acid is at least about 10 nucleotides; preferably at least about 15 nucleotides; and more preferably the length is at least about 20 nucleotides.

In a specific embodiment, the term “standard hybridization conditions” refers to a Tₘ of 55 ° C., and utilizes conditions as set forth above. In a preferred embodiment, the Tₘ is 60° C.; in a more preferred embodiment, the Tₘ is 65° C. In a specific embodiment, “high stringency” refers to hybridization and/or washing conditions at 68° C. in 0.2XSSC, at 42° C. in 50% formamide, 4XSSC, or under conditions that afford levels of hybridization equivalent to those observed under either of these two conditions.

As used herein, the term “oligonucleotide” refers to a nucleic acid, generally of at least 10, preferably at least 15, and more preferably at least 20 nucleotides, preferably no more than 100 nucleotides, that is hybridizable to a genomic DNA molecule, a cDNA molecule, or an mRNA molecule encoding a gene, mRNA, cDNA, or other nucleic acid of interest. Oligonucleotides can be labeled, e.g., with ³²P-nucleotides or nucleotides to which a label, such as biotin, has been covalently conjugated. In one embodiment, a labeled oligonucleotide can be used as a probe to detect the presence of a nucleic acid. In another embodiment, oligonucleotides (one or both of which may be labeled) can be used as PCR primers, either for cloning full length or a fragment of the gene, or to detect the presence of nucleic acids encoding the protein. In a further embodiment, an oligonucleotide of the invention can form a triple helix with a DNA molecule. Generally, oligonucleotides are prepared synthetically, preferably on a nucleic acid synthesizer. Accordingly, oligonucleotides can be prepared with non-naturally occurring phosphoester analog bonds, such as thioester bonds, etc.

The present invention provides antisense nucleic acids (including ribozymes), which may be used to inhibit expression of a target protein of the invention. An “antisense nucleic acid” is a single stranded nucleic acid molecule which, on hybridizing under cytoplasmic conditions with complementary bases in an RNA or DNA molecule, inhibits the latter's role. If the RNA is a messenger RNA transcript, the antisense nucleic acid is a countertranscript or mRNA-interfering complementary nucleic acid. As presently used, “antisense” broadly includes RNA-RNA interactions, RNA-DNA interactions, ribozymes and RNase-H mediated arrest. Antisense nucleic acid molecules can be encoded by a recombinant gene for expression in a cell (e.g, U.S. Pat. No. 5,814,500; U.S. Pat. No. 5,811,234), or alternatively they can be prepared synthetically (e.g., U.S. Pat. No. 5,780,607).

Specific non-limiting examples of synthetic oligonucleotides envisioned for this invention include oligonucleotides that contain phosphorothioates, phosphotriesters, methyl phosphonates, short chain alkyl, or cycloalkl intersugar linkages or short chain heteroatomic or heterocyclic intersugar linkages. Most preferred are those with CH₂—NH—O—CH₂, CH₂—N(CH₃)—O—CH₂, CH₂—O—N(CH₃)—CH₂, CH₂—N(CH₃)—N(CH₃)—CH₂ and O—N(CH₃)—CH₂—CH₂ backbones (where phosphodiester is O—PO₂—O—CH₂). U.S. Pat. No. 5,677,437 describes heteroaromatic olignucleoside linkages. Nitrogen linkers or groups containing nitrogen can also be used to prepare oligonucleotide mimics (U.S. Pat. Nos. 5,792,844 and 5,783,682). U.S. Pat. No. 5,637,684 describes phosphoramidate and phosphorothioamidate oligomeric compounds. Also envisioned are oligonucleotides having morpholino backbone structures (U.S. Pat. No. 5,034,506). In other embodiments, such as the peptide-nucleic acid (PNA) backbone, the phosphodiester backbone of the oligonucleotide may be replaced with a polyamide backbone, the bases being bound directly or indirectly to the aza nitrogen atoms of the polyamide backbone (Nielsen et al., Science 254:1497, 1991). Other synthetic oligonucleotides may contain substituted sugar moieties comprising one of the following at the 2′ position: OH, SH, SCH₃, F, OCN, O(CH₂)ₙNH₂ or O(CH₂)ₙCH₃ where n is from 1 to about 10; C₁ to C₁₀ lower alkyl, substituted lower alkyl, alkaryl or aralkyl; Cl; Br; CN; CF₃; OCF₃; O—; S—, or N-alkyl; O—, S—, or N-alkenyl; SOCH₃ ; SO₂CH₃; ONO₂;NO₂; N₃; NH₂; heterocycloalkyl; heterocycloalkaryl; aminoalkylamino; polyalkylamino; substituted silyl; a fluorescein moiety; an RNA cleaving group; a reporter group; an intercalator; a group for improving the pharmacokinetic properties of an oligonucleotide; or a group for improving the pharmacodynamic properties of an oligonucleotide, and other substituents having similar properties. Oligonucleotides may also have sugar mimetics such as cyclobutyls or other carbocyclics in place of the pentofuranosyl group. Nucleotide units having nucleosides other than adenosine, cytidine, guanosine, thymidine and uridine, such as inosine, may be used in an oligonucleotide molecule.

### Recombinant Expression Systems

A wide variety of host/expression vector combinations (i.e., expression systems) may be employed in expressing the DNA sequences of this invention, particularly in Xenopus oocytes or embryonic cells. Useful expression vectors, for example, may consist of segments of chromosomal, non-chromosomal and synthetic DNA sequences. Suitable vectors include derivatives of SV40 and known bacterial plasmids, e.g., E. coli plasmids col E1, pCR1, pBR322, pMal-C2, pET, pGEX (Smith et al., Gene 67:31-40, 1988), pMB9 and their derivatives, plasmids such as RP4; phage DNAs, e.g., the numerous derivatives of phage 1, e.g., NM989, and other phage DNA, e.g., M13 and filamentous single stranded phage DNA; yeast plasmids such as the 2m plasmid or derivatives thereof; vectors useful in eukaryotic cells, such as vectors useful in insect or mammalian cells; vectors derived from combinations of plasmids and phage DNAs, such as plasmids that have been modified to employ phage DNA or other expression control sequences; and the like. In addition, various tumor cells lines can be used in expression systems of the invention.

Yeast expression systems can also be used according to the invention to express any protein of interest. For example, the non-fusion pYES2 vector (XbaI, SphI, ShoI, NotI, GstXI, EcoRI, BstXI, BamH1, SacI, Kpn1, and HindIII cloning sit; Invitrogen) or the fusion pYESHisA, B, C (XbaI, SphI, Shol, NotI, BstXI, EcoRI, BamH1, SadI, KpnI, and HindIII cloning site, N-terminal peptide purified with ProBond resin and cleaved with enterokinase; Invitrogen), to mention just two, can be employed according to the invention.

Expression of the protein or polypeptide may be controlled by any promoter/enhancer element known in the art, but these regulatory elements must be functional in the host selected for expression. Promoters which may be used to control gene expression include, but are not limited to, cytomegalovirus (CMV) promoter (U.S. Pat. Nos. 5,385,839 and 5,168,062), the SV40 early promoter region (Benoist and Chambon, 1981, Nature 290:304-310), the promoter contained in the 3′ long terminal repeat of Rous sarcoma virus (Yamamoto, et al., Cell 22:787-797, 1980), the herpes thymidine kinase promoter (Wagner et al., Proc. Natl. Acad. Sci. U.S.A. 78:1441-1445, 1981), the regulatory sequences of the metallothionein gene (Brinster et al., Nature 296:39-42, 1982); prokaryotic expression vectors such as the β-lactamase promoter (Villa-Komaroff, et al., Proc. Natl. Acad. Sci. U.S.A. 75:3727-3731, 1978), or the tac promoter (DeBoer, et al., Proc. Natl. Acad. Sci. U.S.A. 80:21-25, 1983); see also “Useful proteins from recombinant bacteria” in Scientific American, 242:74-94, 1980; and promoter elements from yeast or other fungi such as the Gal 4 promoter, the ADC (alcohol dehydrogenase) promoter, PGK (phosphoglycerol kinase) promoter, alkaline phosphatase promoter.

Preferred vectors, particularly for cellular assays in vitro and in vivo, are viral vectors, such as lentiviruses, retroviruses, herpes viruses, adenoviruses, adeno-associated viruses, vaccinia virus, baculovirus, and other recombinant viruses with desirable cellular tropism. Thus, a gene encoding a functional or mutant protein or polypeptide domain fragment thereof can be introduced in vivo, ex vivo, or in vitro using a viral vector or through direct introduction of DNA. Expression in targeted tissues can be effected by targeting the transgenic vector to specific cells, such as with a viral vector or a receptor ligand, or by using a tissue-specific promoter, or both. Targeted gene delivery is described in International Patent Publication WO 95/28494, published October 1995.

Viral vectors commonly used for in vivo or ex vivo targeting and therapy procedures are DNA-based vectors and retroviral vectors. Methods for constructing and using viral vectors are known in the art (see, e.g., Miller and Rosman, BioTechniques, 7:980-990, 1992). Preferably, the viral vectors are replication defective, that is, they are unable to replicate autonomously in the target cell. Preferably, the replication defective virus is a minimal virus, i.e., it retains only the sequences of its genome which are necessary for encapsidating the genome to produce viral particles.

DNA viral vectors include an attenuated or defective DNA virus, such as but not limited to herpes simplex virus (HSV), papillomavirus, Epstein Barr virus (EBV), adenovirus, adeno-associated virus (AAV), and the like. Defective viruses, which entirely or almost entirely lack viral genes, are preferred. Defective virus is not infective after introduction into a cell. Use of defective viral vectors allows for administration to cells in a specific, localized area, without concern that the vector can infect other cells. Thus, a specific tissue can be specifically targeted. Examples of particular vectors include, but are not limited to, a defective herpes virus 1 (HSV 1) vector (Kaplitt et al., Molec. Cell. Neurosci. 2:320-330, 1991), defective herpes virus vector lacking a glyco-protein L gene (Patent Publication RD 371005 A), or other defective herpes virus vectors (International Patent Publication No. WO 94/21807, published Sep. 29, 1994; International Patent Publication No. WO 92/05263, published Apr. 2, 1994); an attenuated adenovirus vector, such as the vector described by Stratford-Perricaudet et al. (J. Clin. Invest. 90:626-630, 1992; see also La Salle et al, Science 259:988-990, 1993); and a defective adeno-associated virus vector (Samulski et al., J. Virol. 61:3096-3101, 1987; Samulski et al., J. Virol. 63:3822-3828, 1989; Lebkowski et al., Mol. Cell. Biol. 8:3988-3996, 1988).

Various companies produce viral vectors commercially, including but by no means limited to Avigen, Inc. (Alameda, Calif.; AAV vectors), Cell Genesys (Foster City, Calif.; retroviral, adenoviral, AAV vectors, and lentiviral vectors), Clontech (retroviral and baculoviral vectors), Genovo, Inc. (Sharon Hill, Pa.; adenoviral and AAV vectors), Genvec (adenoviral vectors), IntroGene (Leiden, Netherlands; adenoviral vectors), Molecular Medicine (retroviral, adenoviral, AAV, and herpes viral vectors), Norgen (adenoviral vectors), Oxford BioMedica (Oxford, United Kingdom; lentiviral vectors), and Transgene (Strasbourg, France; adenoviral, vaccinia, retroviral, and lentiviral vectors).

### Microarrays

In a preferred embodiment the present invention makes use of microarrays for identifying the large numbers of genes involved in embryonic development and related processes such as cell differentiation, and for fingerprinting expression patterns.

In one embodiment, microarrays are produced by hybridizing detectably labeled polynucleotides representing the cDNA sequences from an embryonic expression library (e.g., fluorescently labeled cDNA synthesized from total mRNA) to a microarray. A microarray is a surface with an ordered array of binding (e.g., hybridization) sites for products of many of the genes in the genome of a cell or organism, preferably most or almost all of the genes. Microarrays can be made in a number of ways, of which several are described below. However produced, microarrays share certain characteristics: The arrays are reproducible, allowing multiple copies of a given array to be produced and easily compared with each other. Preferably the microarrays are small, usually smaller than 5 cm², and they are made from materials that are stable under binding (e.g. nucleic acid hybridization) conditions. A given binding site or unique set of binding sites in the microarray will specifically bind the product of a single gene. Although there may be more than one physical binding site (hereinafter “site”) per specific MRNA, for the sake of clarity the discussion below will assume that there is a single site.

It will be appreciated that when cDNA complementary to the RNA of a cell is made and hybridized to a microarray under suitable hybridization conditions, the level of hybridization to the site in the array corresponding to any particular gene will reflect the prevalence in the cell of mRNA transcribed from that gene. For example, when detectably labeled (e.g., with a fluorophore) cDNA complementary to the total cellular mRNA is hybridized to a microarray, the site on the array corresponding to a gene (i.e., capable of specifically binding the product of the gene) that is not transcribed in the cell will have little or no signal (e.g., fluorescent signal), and a gene for which the encoded mRNA is prevalent will have a relatively strong signal.

In preferred embodiments, cDNAs from Xenopus clones are hybridized to the binding sites of the microarray. The cDNA derived from each of the different Xenopus clones are differently labeled so that they can be distinguished. In one embodiment, for example, one clone, the cDNA may be synthesized using a fluorescein-labeled dNTP, and cDNA from a second clone synthesized using a rhodamine-labeled dNTP. When a number of cDNAs are mixed and hybridized to the microarray, the relative intensity of signal from each cDNA set is determined for each site on the array, and any relative difference in abundance of a particular mRNA detected.

The use of a two-color fluorescence labeling and detection scheme to define alterations in gene expression has been described, e.g., in Shena et al., (Science 1995 270:467-470), which is incorporated by reference in its entirety for all purposes. An advantage of using cDNA labeled with two different fluorophores is that a direct and internally controlled comparison of the mRNA levels corresponding to each arrayed gene in two cell states can be made, and variations due to minor differences in experimental conditions (e.g., hybridization conditions) will not affect subsequent analyses. However, it will be recognized that it is also possible to use cDNA from a single cell, and compare, for example, the absolute amount of a particular mRNA.

### Preparation of Microarrays

Microarrays are known in the art and consist of a surface to which probes that correspond in sequence to gene products (e.g., cDNAs, mRNAs, cRNAs, polypeptides, and fragments thereof), can be specifically attached or bound at a known position.

In one embodiment, the microarray is an array (i.e., a matrix) in which each position represents a discrete binding site for a product encoded by a gene (e.g., a protein or RNA), and in which binding sites are present for products of most or almost all of the genes in the organism's genome. In a preferred embodiment, the “binding site” (hereinafter, “site”) is a nucleic acid or nucleic acid analogue to which a particular cognate cDNA can specifically hybridize. The nucleic acid or analogue of the binding site can be, e.g., a synthetic oligomer, a full-length cDNA, a less-than full length cDNA, or a gene fragment.

Although in a preferred embodiment the microarray contains binding sites for products of all or almost all genes in the target organism's genome, such comprehensiveness is not necessarily required. Usually the microarray will have binding sites corresponding to at least about 50% of the genes in the genome, often at least about 75%, more often at least about 85%, even more often more than about 90%, and most often at least about 99%. Preferably, the microarray has full length genes involved in embryonic development or cell differentiation. In the context of microarrays, a “gene” is identified as an open reading frame (ORF) of preferably at least 50, 75, or 99 amino acids from which a messenger RNA is transcribed in the organism (e.g., if a single cell) or in some cell in a multicellular organism. The number of genes in a genome can be estimated from the number of mRNAs expressed by the organism, or by extrapolation from a well-characterized portion of the genome. When the genome of the organism of interest has been sequenced, the number of ORFs can be determined and mRNA coding regions identified by analysis of the DNA sequence. For example, the Saccharomyces cerevisiae genome has been completely sequenced and is reported to have approximately 6275 open reading frames (ORFs) longer than 99 amino acids. Analysis of these ORFs indicates that there are 5885 ORFs that are likely to specify protein products (Goffeau et al., 1996 Science 274:546-567. In contrast, the human genome is estimated to contain approximately 10⁵ genes.

### Preparing Nucleic Acids for Microarrays

As noted above, the “binding site” to which a particular cognate cDNA specifically hybridizes is usually a nucleic acid or nucleic acid analogue attached at that binding site. In one embodiment, the binding sites of the microarray are DNA polynucleotides corresponding to at least a portion of each gene or preferably the full-length gene in an organism's genome. These DNAs can be obtained by, e.g., polymerase chain reaction (PCR) amplification of gene segments from genomic DNA, cDNA (e.g., by RT-PCR), or cloned sequences. PCR primers are chosen, based on the known sequence of the genes or cDNA, that result in amplification of unique fragments (i.e. fragments that do not share more than 10 bases of contiguous identical sequence with any other fragment on the microarray). Computer programs are useful in the design of primers with the required specificity and optimal amplification properties. See, e.g., Oligo version 5.0 (National Biosciences). In the case of binding sites corresponding to very long genes, it will sometimes be desirable to amplify segments near the 3′ end of the gene so that when oligo-dT primed cDNA probes are hybridized to the microarray, less-than-full length probes will bind efficiently. Typically each gene or gene fragment on the microarray will be between about 31 and 815 bp, more typically between about 148 and 815 in length. PCR methods are well known and are described, for example, in Innis et al. eds., 1990, PCR Protocols: A Guide to Methods and Applications, Academic Press Inc. San Diego, Calif., which is incorporated by reference in its entirety for all purposes. It will be apparent that computer controlled robotic systems are useful for isolating and amplifying nucleic acids.

An alternative means for generating the nucleic acid for the microarray is by synthesis of synthetic polynucleotides or oligonucleotides, e.g., using N-phosphonate or phosphoramidite chemistries (Froehler et al., Nucleic Acid Resl4:5399-5407, 1986; McBride et al., Tetrahedron Lett. 24:245-248, 1983). Synthetic sequences are between about 15 and about 500 bases in length, more typically between about and about 50 bases. In some embodiments, synthetic nucleic acids include non-natural bases, e.g., inosine. As noted above, nucleic acid analogues may be used as binding sites for hybridization. An example of a suitable nucleic acid analogue is peptide nucleic acid (see, e.g., Egholm et al., Nature 365:566-568, 1993; see also U.S. Pat.No. 5,539,083).

In an alternative embodiment, the binding (hybridization) sites are made from phage clones of genes, expressed sequence tags or inserts therefrom. In yet another embodiment, the polynucleotide of the binding sites is RNA.

### Attaching Nucleic Acids to the Solid Surface

The nucleic acid or analogue are attached to a solid support, which may be made from glass, plastic (e.g., polypropylene, nylon), polyacrylamide, nitrocellulose, or other materials. A preferred method for attaching the nucleic acids to a surface is by printing on glass plates, as is described generally by Schena et al., Science 270:467-470, 1995. This method is especially useful for preparing microarrays of cDNA. See also DeRisi et al., Nature Genetics 14:457-460, 1996,; Shalon et al., Genome Res. 6:639-645, 1996; and Schena et al., Proc. Natl. Acad. Sci. USA 93:10539-11286, 1995. Each of the aforementioned articles is incorporated by reference in its entirety for all purposes.

A preferred method of making microarrays is by use of an inkjet printing process to bind genes or oligonucleotides directly on a solid phase, as described, e.g., in U.S. Pat. No. 5,965,352 which is incorporated by reference herein in its entirety.

Other methods for making microarrays, e.g., by masking (Maskos and Southern, Nuc. Acids Res. 20:1679-1684, 1992, ), may also be used. In principal, any type of array, for example, dot blots on a nylon hybridization membrane (see Sambrook et al., Molecular Cloning A Laboratory Manual (2nd Ed.), Vol. 1-3, Cold Spring Harbor Laboratory, Cold Spring Harbor, N.Y., 1989, which is incorporated in its entirety for all purposes), could be used, although, as will be recognized by those of skill in the art, very small arrays will be preferred because hybridization volumes will be smaller.

### Generating Labeled Probes

Methods for preparing total and poly(A)⁺ RNA are well known and are described generally in Sambrook et al., supra. In one embodiment, RNA is extracted from cells of the various types of interest in this invention using guanidinium thiocyanate lysis followed by CsCl centrifuigation (Chirgwin et al., Biochemistry 18:5294-5299, 1979,. Poly(A)⁺ RNA is selected by selection with oligo-dT cellulose (see Sambrook et al., supra). Cells of interest include embryonic cells.

Labeled cDNA is prepared from mRNA by oligo dT-primed or random-primed reverse transcription, both of which are well known in the art (see e.g., Klug and Berger, Methods Enzymol. 152:316-325, 1987). Reverse transcription may be carried out in the presence of a dNTP conjugated to a detectable label, most preferably a fluorescently labeled dNTP. Alternatively, isolated mRNA can be converted to labeled antisense RNA synthesized by in vitro transcription of double-stranded cDNA in the presence of labeled dNTPs (Lockhart et al., Nature Biotech. 14:1675, 1996, which is incorporated by reference in its entirety for all purposes). cDNA or RNA probe can be synthesized in the absence of detectable label and may be labeled subsequently, e.g., by incorporating biotinylated dNTPs or rNTP, or some similar means (e.g., photo-cross-linking a psoralen derivative of biotin to RNAs), followed by addition of labeled streptavidin (e.g., phycoerythrin-conjugated streptavidin) or the equivalent.

When fluorescently-labeled probes are used, many suitable fluorophores are known, including fluorescein, lissamine, phycoerythrin, rhodamine (Perkin Elmer Cetus), Cy2, Cy3, Cy3.5, Cy5, Cy5.5, Cy7, FluorX (Amersham) and others (see, e.g., Kricka, Nonisotopic DNA Probe Techniques, 1992, Academic Press San Diego, Calif.). It will be appreciated that pairs of fluorophores are chosen that have distinct emission spectra so that they can be easily distinguished.

In one embodiment, labeled cDNA is synthesized by incubating a mixture containing 0.5 mM dGTP, dATP and dCTP plus 0.1 mM dTTP plus fluorescent deoxyribonucleotides (e.g., 0.1 mM Rhodamine 110 UTP (Perken Elmer Cetus) or 0.1 mM Cy3 dUTP (Amersham)) with reverse transcriptase (e.g., SuperScript.TM. II, LTI Inc.) at 42° C. for 60 min.

### Hybridization to Microarrays

Nucleic acid hybridization and wash conditions are chosen so that the probe “specifically binds” or “specifically hybridizes” to a specific array site, i.e., the probe hybridizes, duplexes or binds to a sequence array site with a complementary nucleic acid sequence but does not hybridize to a site with a non-complementary nucleic acid sequence. As used herein, one polynucleotide sequence is considered complementary to another when, if the shorter of the polynucleotides is less than or equal to 25 bases, there are no mismatches using standard base-pairing rules or, if the shorter of the polynucleotides is longer than 25 bases, there is no more than a 5% mismatch. Preferably, the polynucleotides are perfectly complementary (no mismatches). It can easily be demonstrated that specific hybridization conditions result in specific hybridization by carrying out a hybridization assay including negative controls (see, e.g., Shalon et al . supra, and Chee et al., Science 274:610-614, 1996.

Optimal hybridization conditions will depend on the length (e.g., oligomer versus polynucleotide greater than 200 bases) and type (e.g., RNA, DNA, PNA) of labeled probe and immobilized polynucleotide or oligonucleotide. General parameters for specific (i.e., stringent) hybridization conditions for nucleic acids are described in Sambrook et al., supra, and in Ausubel et al., Current Protocols in Molecular Biology, Greene Publishing and Wiley-Interscience, New York, 1987, which is incorporated in its entirety for all purposes. When the cDNA microarrays of Schena et al. are used, typical hybridization conditions are hybridization in 5×SSC plus 0.2% SDS at 65° C. for 4 hours followed by washes at 25° C. in low stringency wash buffer (1×SSC plus 0.2% SDS) followed by 10 minutes at 25° C. in high stringency wash buffer (0.1×SSC plus 0.2% SDS) (Shena et al., Proc. NatI. Acad. Sci. USA, 93:10614,1996,). Useful hybridization conditions are also provided in, e.g., Tijessen, 1993, Hybridization With Nucleic Acid Probes, Elsevier Science Publishers B.V. and Kricka, 1992, Nonisotopic DNA Probe Techniques, Academic Press San Diego, Calif.

### Signal Detection and Data Analysis

When fluorescently labeled probes are used, the fluorescence emissions at each site of a transcript array can be, preferably, detected by scanning confocal laser microscopy. In one embodiment, a separate scan, using the appropriate excitation line, is carried out for each of the two fluorophores used. Alternatively, a laser can be used that allows simultaneous specimen illumination at wavelengths specific to the two fluorophores and emissions from the two fluorophores can be analyzed simultaneously (see Shalon et al., supra, which is incorporated by reference in its entirety for all purposes). In a preferred embodiment, the arrays are scanned with a laser fluorescent scanner with a computer controlled X-Y stage and a microscope objective. Sequential excitation of the two fluorophores is achieved with a multi-line, mixed gas laser and the emitted light is split by wavelength and detected with two photomultiplier tubes. Fluorescence laser scanning devices are described in Schena et al., Genome Res. 6:639-645, 1996 and in other references cited herein. Alternatively, the fiber-optic bundle described by Ferguson et al., Nature Biotech. 14:1681-1684, 1996, may be used to monitor mRNA abundance levels at a large number of sites simultaneously.

Signals are recorded and, in a preferred embodiment, analyzed by computer, e.g., using a 12 bit analog to digital board. In one embodiment the scanned image is despeckled using a graphics program (e.g., Hijaak Graphics Suite) and then analyzed using an image gridding program that creates a spreadsheet of the average hybridization at each wavelength at each site. If necessary, an experimentally determined correction for “cross talk” (or overlap) between the channels for the two fluors may be made. For any particular hybridization site on the transcript array, a ratio of the emission of the two fluorophores can be calculated. The ratio is independent of the absolute expression level of the cognate gene, but is useful for genes whose expression is significantly modulated by drug administration, gene deletion, or any other tested event.

According to the method of the invention, the relative abundance of an mRNA in two cells, cell lines or Xenopus clones are scored as a perturbation and its magnitude determined (i.e., the abundance is different in the two sources of mRNA tested), or as not perturbed (i.e., the relative abundance is the same). As used herein, a difference between the two sources of RNA of at least a factor of about 25% (RNA from one source is 25% more abundant in one source than the other source), more usually about 50%, even more often by a factor of about 2 (twice as abundant), 3 (three times as abundant) or 5 (five times as abundant) is scored as a perturbation. Present detection methods allow reliable detection of difference of an order of about 3-fold to about 5-fold, but more sensitive methods are expected to be developed.

Preferably, in addition to identifying a perturbation as positive or negative, it is advantageous to determine the magnitude of the perturbation. This can be carried out, as noted above, by calculating the ratio of the emission of the two fluorophores used for differential labeling, or by analogous methods that will be readily apparent to those of skill in the art.

### Uses of the Nucleic Acid Arrays

The embryonic gene expression nucleic acid arrays of the invention have a number of potential uses, all of which turn on the ability to detect differences of expression of gene products as a result of some change between cells.

“Changes between cells” refers to differences in time, location, or environment, which are reflected in differential gene expression. For convenience, the reference cells are referred to as “control” cells; the cells that have undergone a change relative to the control cells are “sample” cells. Naturally, these terms are relatively arbitrary as applied to the cells. However, in general usage control cells are cells at an earlier time or that have not undergone any environmental changes.

### Gene Expression in Development

In one embodiment, the embryonic nucleic acid arrays permit identification of gene expression during development. Thus, differences in gene expression can be correlated with time or stage of development, or with cellular differentiation into different tissues. This information permits identification of gene products associated with embryonic development, e.g., by cloning and sequencing genes whose expression varies in interesting ways during the development process. The array also establishes a genetic “fingerprint”, i.e., a pattern of gene expression that provides information about the developmental process even in the absence of specific sequence information.

This developmental fingerprint has important implications for prenatal testing, particularly in humans. At present, prenatal genotyping consists primarily if not exclusively of karyotyping embryonic or fetal cells, e.g., obtained from amniotic fluid. These methods are both crude and dangerous. Crude, because karyotyping only permits identification of few abnormalities associated with polyploidy. Dangerous because the procedures employed to obtain the fetal cells, such as amniocentesis, can cause harm to the fetus.

By combining PCR with analysis on the embryonic expression arrays of the invention, one can amplify the expressed genes from a single fetal cell, which might be obtained from maternal blood or some other non-invasive source (see, e.g., Huber et al, Prenat. Diagn. 2000, 20:479; Campagnoli et al., Jun. 21, 1999 at the 18th Meeting of the International Fetal Medicine & Surgery Society; Campagnoli et al., Dec. 4, 1999 at the 41st Annual Meeting And Exposition of the American Society of Hematology, Abstr. #157). The expressed genes from these cells can be evaluated on the embryonic expression nucleic acid array for appropriate expression patterns. The presence or absence of key genes at a particular stage of fetal development will provide important information about fetal viability, the presence of possible genetic defects, and other information that will permit true and effective genetic counseling of parents, as well as warn of possible adverse outcomes, thus permitting the mother to adopt changes calculated to negate these outcomes.

Given the great degree of sequence conservation and homology of developmental genes between members of otherwise disparate species, work done on the Xenopus arrays specifically exemplified infra provide the information about corresponding expression patterns in mammals, and particularly humans. Moreover, human embryonic libraries (Adjaye et al., Gene 1999, 237:373; Adjaye et al., Genomics 1997, 46:337; Daniels et al., Hum. Reprod. 1997 12:2251) are available and can be adapted to the practice of the invention.

### Determining Gene or Protein Function

In another embodiment, the expression patterns of genes of the invention permit evaluation of gene function. These “cluster” patterns are associated with development, differentiation, or some stimulus, e.g., contact with a growth factor. By establishing expression patterns in response to known modulators and factors, e.g., growth factor, apoptotic factors, cytokines and lymphokines, hormones, neurotransmitters, etc., the nucleic acid arrays of the invention provide a powerful tool for studying these processes in the context of development. Furthermore, function of unknown gene products can be evaluated by comparing expression patterns resulting from exposure to these gene products (proteins or nucleic acids encoding them) with established expression patterns. The unknown gene products can be introduced into embryonic cells (including oocytes) as nucleic acid vectors, or as proteins. Because protein function is highly conserved, particularly in embryonic cells, the unknown gene product need not be from Xenopus.

As noted above, expression patterns of known (sequenced) gene products and unknown gene products, or a combination of the two, can provide important information about the function of a known or unknown biomolecule, including identification of genes regulated by the biomolecule.

### Toxin and Drug Testing

In a particularly preferred embodiment, the embryonic expression nucleic acid arrays of the invention provide a platform for toxicity or drug testing. At present, live animals serve as subjects for evaluating toxic compounds, pollutants, or drugs. Because they are particularly sensitive to toxins, embryonic organisms are often preferred for many tests. Thus the expression arrays of the invention can substitute or replace live animals for many testing purposes. Furthermore, because test outcomes turn on detecting differential gene expression that lead to physiological or anatomical manifestations of toxicity, rather than waiting for the actual manifestation of these changes, it is much more time effective.

For example, presently, water quality tests involve contacting aquatic eggs or newly hatched fish or frogs with water to be tested. The health, viability, and presence of mutations are evaluated. Changes in embryonic gene expression can be detected using the arrays because toxins elicit specific expression patterns (e.g., such as metallothionein in response to heavy metals).

In addition to testing water, other environmental pollutants or toxins can be tested using the systems of the invention. These include air quality, solid waste contaminants, and the like.

A related embodiment of the invention tests toxicity of drugs or drug candidates, i.e., as an auxiliary, supplement, or replacement of animal testing.

In both toxicity and drug testing, expression patterns observed in response to known toxins or drugs establish the response patterns. Expression patterns observed in response to unknown samples or drugs can be compared to the established response patterns to identify toxicity.

### EXAMPLES

The present invention is also described by means of particular examples. However, the use of such examples anywhere in the specification is illustrative only and in no way limits the scope and meaning of the invention or of any exemplified term. Likewise, the invention is not limited to any particular preferred embodiments described herein. Indeed, many modifications and variations of the invention will be apparent to those skilled in the art upon reading this specification and can be made without departing from its spirit and scope. The invention is therefore to be limited only by the terms of the appended claims along with the fall scope of equivalents to which the claims are entitled.

### Clone Preparation and Analysis

Plasmids from the Xenopus early gastrula library of Weinstein et al. (Development 1997, 124:4235) were plated at low density to avoid cross contamination. Individual clones were randomly picked by hand and grown overnight in 1.2 ml Terrific Broth in eight Qiagen 96 well deep well blocks, for a total of 8 ×96 =768 clones.

The plasmids were purified using a Qiagen Turbo 96 kit on a Qiagen Biorobot 9600, and eluted to a 150 μl volume. The approximate DNA concentration of each clone was determined by random sampling of the resulting plasmids to be 0.2 μg/μl.

The 768 different clones were then sequenced from the 5′-end on ABI 3700 sequencers using Big Dye chemistry with a sequencing primer, designated SP6-22 (SEQ ID NO:1), having the nucleotide sequence: 5′-CTTGATTTAGGTGACACTATAG-3′ (SP6-22; SEQ ID NO:1). The sequences were analyzed and organized using the automated sequence annotation tool MAGPIE (Gaasterland and Sensen, Trends Genet. 1996, 12:76; Caasterland and Sensen, Biochimie 1996, 78:302). The Xenopus sequences from each of the sequenced clones are provided in Appendix 1. These clones were organized into eight blocks (S10-1 through S10-8) of 96 clones corresponding to the 96 well block in which the clones were incubated. The Table in Appendix 2 from MAGPIE summarizes all the information gathered together for the 768 clones and includes a specific identification number, the size in base-pairs and the description for each gene. The Table shows that a number of the novel genes were identified from the clones and may play an important role in the process of embryiogenesis.

The results of the sequence analysis are summarized below in Table 1. Specifically, Table 1 shows the number of clones in each of the 96 well blocks (S10-1 through S10-8) for which “hits” (i e., homologous sequences) were found in the NCBI EST database (BlastEST), in the NCBI protein and nucleic acid databases (BLASTX and BLASTN, respectively) and FastaCHIP with various levels of statistical significance (E≦10⁻³⁵, E≦10⁻²⁵, and E≦10⁻⁵)

TABLE I

| Clones: | S10-1 | S10-2 | S10-3 | S10-4 | S10-5 | S10-6 | S10-7 | S10-8 | Total |
|---|---|---|---|---|---|---|---|---|---|
| BlastEST | | | | | | | | | |
| E ≦ 10⁻³⁵ | 37 | 31 | 32 | 41 | 39 | 30 | 29 | 32 | 271 |
| E ≦ 10⁻¹⁵ | 6 | 3 | 10 | 4 | 7 | 2 | 6 | 5 | 43 |
| E ≦ 10⁻⁵ | 30 | 27 | 26 | 21 | 28 | 30 | 21 | 23 | 206 |
| No hits | 23 | 35 | 28 | 30 | 22 | 34 | 40 | 36 | 248 |
| BlastX | | | | | | | | | |
| E ≦ 10⁻³⁵ | 42 | 34 | 46 | 39 | 40 | 39 | 42 | 38 | 320 |
| E ≦ 10⁻¹⁵ | 10 | 13 | 10 | 12 | 16 | 8 | 12 | 12 | 93 |
| E ≦ 10⁻⁵ | 12 | 17 | 10 | 10 | 15 | 12 | 11 | 7 | 94 |
| No hits | 32 | 32 | 30 | 35 | 25 | 37 | 31 | 39 | 261 |
| Blastn | | | | | | | | | |
| E ≦ 10⁻³⁵ | 34 | 30 | 39 | 33 | 36 | 37 | 35 | 33 | 277 |
| E ≦ 10⁻²⁵ | 5 | 2 | 5 | 5 | 6 | 2 | 1 | 6 | 32 |
| E ≦ 10⁻⁵ | 21 | 15 | 16 | 18 | 17 | 17 | 17 | 15 | 136 |
| No hits | 36 | 49 | 36 | 40 | 37 | 40 | 43 | 42 | 323 |
| FastaCHIP | | | | | | | | | |
| E ≦ 10⁻³⁵ | 70 | 72 | 79 | 76 | 44 | 67 | 71 | 61 | 540 |
| E ≦ 10⁻²⁵ | 9 | 7 | 3 | 1 | 9 | 6 | 15 | 13 | 63 |
| E ≦ 10⁻⁵ | 1 | 1 | 2 | 3 | 5 | 3 | 5 | 5 | 25 |
| No hits | 16 | 16 | 12 | 16 | 38 | 20 | 5 | 17 | 140 |

In more detail, of the 768 sequenced clones, 596 (78%) had sequence homologies to at least one other sequence with Expect values (also referred to as “E-values” or “E”) less than 10⁻⁵. Because the E-value is a statistical parameter, calculated by the BLAST algorithm, which represents the probability that a sequence alignment will occur purely by chance, these alignments were determined to be statistically significant alignments representing actual homologous and related sequences.

The BLASTX algorithm was also used to identify protein sequences in the NCBI protein database that were homologous to amino acid sequences translated in all possible reading frames of the sequenced clones. By determining the point in the protein sequences where regions of homology begins, it was determined that 30% of the clones having statistically significant BLAST alignments are full length clones that include the codon for the start methionine of an actual gene. In particular, in these alignments, the average “query sequence” (i.e., the protein sequence predicted for the particular clone) began 130±96 codons upstream of the homology region. In contrast, for 19% of the clones with statistically significant BLASTX alignments, the homology region began much further downstream (an average of 226±194 codons) of the aligning protein sequence's start codon. Thus, these clones were identified as partial clones. Conclusions could not be drawn from the remaining 50% of the clones with statistically significant BLAST alignments since these clones had very low levels of sequence homology.

The remaining 172 sequenced clones had no statistically significant sequence alignments in any of the databases searched. Accordingly, the sequences of these clones are expected to extend into the coding region of their corresponding gene and are not expected to be part of the gene's 5′-untranslated region.

A number of genes that aligned to the clone sequences were genes from plants and/or fungi for which orthologs had not previously been identified in vetebrates or other animal species. Other clones aligned with genes that had previously been identified in lower animals (i.e., in vertebrates) but had not been identified in any vertebrate species.

The individual clones are grouped into several general categories according to the classes of proteins with which they exhibited sequence homology. These categories include: secreted factors, membrane bound proteins, signal transduction, transcription factors, structural proteins, and cellular metabolism. The remaining clones sequenced could not be assigned to a specific category because of their low homology to known sequences.

### Preparation and Hybridization to Xenopus cDNA Microarrays

Polylysine coated slides were prepared according to standard protocols (DeRisi et al. supra, and clones were arrayed thereon using a Stanford type arrayer with quill type pins manufactured according to specifications. Eight plates of random clones prepared as described in Example 1, above, were printed in duplicate, along with 96 previously characterized clones. Pursuant to standard protocols (DeRisi et al, FEBS Lett. 2000, 470:156, Lashkari et al., Proc. Natl. Acad. Sci. USA 1997, 94:13057; DeRisi and Iyer, Curr. Opin. Oncol. 1999, 11:76), the arrays were stored at room temperature for one week before further processing.

cDNA probes for hybridization to the microarrays were prepared from either polyA⁺ selected RNA or total RNA according to standard protocols (DeRisi et al, supra). Briefly, 1 to 2 μg of polyA⁺ RNA or 15 μg of total RNA were used in Reverse Transcriptase (RT) reactions primed with oligo(dT)₁₈₋₂₂ using Superscript II (Gibco/BRL) according to the manufacturer's instructions in 30 μL final volume. Either Cy3 dUTP or Cy5 dUTP (Amersham) was included in the reaction at 15 mM concentration. Unlabeled dTTP was also included in the reaction at 10 mM concentration, while dATP, dGTP and dCTP were present at 25 mM concentrations.

The reactions were incubated at 42° C. for two hours, followed by RNA degradation by the addition of 15 μL of 0.1 N sodium hydroxide and incubation at 70° C. for ten minutes, followed by the addition 15 μL of 0.1N HCl to neutralize the sodium hydroxide. The preparations were then diluted to a volume of 500 ,μL with TE prior. Unincorporated nucleotides and dyes were next removed by adding poly(dA) and filtering the preparations in Microcon-30 filters. The samples were subsequently washed twice in 500 μL TE before being combined, concentrated and dried. The combined samples were resuspended in 15 μL of 3×SSC containing 0.3% SDS and filtered through a pre-wet Millipore filter to remove particulates.

For hybridization, the probes were heated to 100° C. for three minutes and applied to the Xenopus cDNA microarray, covered with a 22×22 mm glass coverslip (Fisher #12-542B) and sealed in a hybridization chamber (Stanford). The samples were incubated overnight at 65° C. Following hybridization, the microarray was washed three times at room temperature. Specifically, the microarray was washed, first, for ten minutes in 1×SSC containing 3% SDS, followed by washing for ten minutes in 0.2×SSC, and a ten minute wash in 0.05×SSC. The slides were then dried by centrifugation and stored in the dare at room temperature before scanning.

The microarrays were scanned using a ScanArray 3000 confocal laser scanner (General Scanning, Inc.) to generate two 16 bit greyscale TIFF images corresponding to fluorescence observed on the microarray from the Cy3 and Cy5 labels, respectively. The TIFF images analyzed using Scanalyze version 2.44 (M. Einsen, Stanford University; available from the URL: <http://rana.Stanford.EDU/software> and gridded according to software instructions. The results were mapped to the sequence information generated in Example 1, above.

### Gene Expression From Different Embryonic Stages

Gene expression was compared from pre-MBT Xenopus embryos to post MBT gastrula stage embryos using the microarrays described in Example 2, above. Because the mRNAs expressed during the first hours of Xenopus development are maternal mRNAs transcribed during oogenesis, changes in mRNA expression between two cell types are indicative of genes involved in embryo development.

For these studies, RNA was isolated from 32 cell embryos (stage 6) and early gastrula stage embryos (stage 11). The RNA from each embryo was oligo(dT) selected to enrich for mRNA and 1-2 μL of polyA⁺ mRNA from the each of two embryos was differentially labeled with Cy3 or Cy5 dUTP, respectively, using reverse transcriptase, as described above in Example 2. The resulting cDNAs were hybridized onto microarrays containing the Xenopus clones described in Example 1, above, according to the hybridization methods described in Example 2. To minimize experimental errors resulting from differences in dye incorporation, the experiment was repeated using reverse labeling. Thus, a first hybridization experiment was performed using Cy3 labeled polyA⁺ mRNA from 32 cell embryos and Cy5 labeled polyA⁺ from early gastrula stage embryos, and a second, otherwise identical hybridization experiment was performed with Cy5 labeled polyA⁺ mRNA from 32 cell embryos and Cy3 labeled polyA⁺ mRNA from early gastrula stage embryos. Thus four data points were generated for each clone on the microarray (i.e., one data point for each of the two different labels for mRNA extracted from each of the two embryos). Of the 3456 data points generated, approximately 2100 of these were discarded as their intensities was less than twice the standard error of the average background signal in both channels.

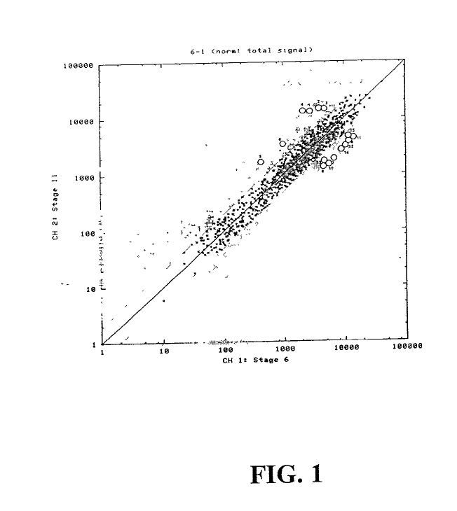

A typical plot of fluorescence intensity values from a Xenopus microarray is provided in FIG. 1. Specifically, the scatter plot in FIG. 1 compares, for each clone on the microarray, the fluorescence intensity of the corresponding cDNA from the stage 6 (horizontal axis) and stage 11 (vertical axis) embryo cDNA samples hybridized to the microarray. Genes that lie on or near the diagonal (i.e., between the two dashed lines) in FIG. 1 are expressed at the same or similar levels in both embryos. However, numerous genes were also identified that are either upregulated or downregulated in the 32 cell stage embryos. The genes can be readily seen in FIG. 1 since they correspond to points that lie away from the diagonal.

In more detail, among the 768 clones on the microarray, 123 (16%) correspond to genes that were upregulated by a factor of two or more in gastrula stage embryos relative to the 32 cell embryo. 100 (13%) of the genes were downregulated by a factor of at least two. The remaining clones exhibited much lower changes in expression (less than two-fold) from 32 cell to gastrula stage embryos.

The results of these experiments demonstrate the utility of microarrays for rapidly identifying large numbers of genes involved in embryonic development and related processes such as cell differentiation. The results also identify particular genes that are activated during such processes and are therefore useful, e.g., for diagnosing developmental disorders and for “fingerprinting” or identifying different types of embryonic cells. Such genes, as well as microarrays with probes to detect expression of such genes (including the particular microarrays described in these examples) are therefore within the scope of the present invention.

### PCR Analysis

To confirm and evaluate the results obtained with microarrays, genes were selected from both up-regulated and down-regulated clones for expression analysis by more specific PCR techniques. Specifically, PCR primers were designed using the Primer3 algorithm (Whitehead Institute) to amplify those clones that were up- or down-regulated by a factor of two or more in all experiments with a standard deviation that less than 5%. Among these clones, the top ten up-regulated and the top ten down-regulated genes were selected for quantitation by RT-PCR as described by Wilson and Hemmati-Brivanlou, 1995.

To ensure that PCR analysis was performed in the linear range, the number of PCR cycles was varied from 15 to 25. The PCR produces were separated on 6% non-denaturing polyacrylamide gels and exposed and examined on a Molecular Dynamics Phosphoimager to ensure a linear readout of the radioactive signal. In order to normalize the signal between the two samples, the ubiquitous protein histone H4 was also amplified.

The results from this PCR analyses are presented below in Table 2. Specifically, this table indicates, for each clone that was analyzed by microarray hybridization and RT-PCR, the average ratio of expression in pre-MBT vs. gastrula stage Xenopus embryos. The results obtained for 80% of the genes analyzed by PCR correlate perfectly with data obtained from microarrays. However, the magnitude of the change observed by the PCR based approach did not always correspond with that observed on microarrays. In some cases, the differences were small (e.g., for the clones S10-1-E7, S10-1-B11, S10-8C11 and S10-1-H7) and fell within 1-2 standard deviations of the values observed on microarrays. However, in other cases (e.g., for the clones S10-4-C2 and S10-4-C1) a much greater difference in expression was observed by PCR. In other cases, the magnitude of the change measured by RT-PCR was actually less than that measured by microarrays (e.g., for the clones S10-2-B10, S10-8-H10, S10-2-F11 and S10-2-E7), and in a few cases little or no change in expression was observed by RT-PCR (e.g., the clones S10-4-D3 and S10-6-G4). In at least on case, the direction of change observed by RT-PCR was opposite that observed using microarray analysis (e.g., for the clones S10-2-E7).

TABLE 2

| | MICROARRAY | | RT-PCR | |
|---|---|---|---|---|
| Clone | Avg. Ratio | Std. Dev. | Avg. Ratio | Std. Dev. |
| S10-1-E7 | 0.18 | 0.01 | 0.16 | 0.01 |
| S10-2-H8 | 0.19 | 0.05 | -failed- | |
| S10-2-B10 | 0.21 | 0.05 | 0.57 | 0.02 |
| S10-8-H10 | 0.26 | 0.07 | 0.58 | 0.04 |
| S10-2-F11 | 0.26 | 0.08 | 0.47 | 0.07 |
| S10-1-B11 | 0.29 | 0.06 | 0.28 | 0.01 |
| S10-8-C11 | 0.29 | 0.08 | 0.21 | 0.01 |
| S10-4-D3 | 0.28 | 0.08 | 0.97 | 0.04 |
| S10-8-A11 | 0.3 | 0.07 | 0.70 | 0.01 |
| S10-8-D4 | 0.33 | 0.06 | -failed- | |
| S10-2-E7 | 5.04 | 1.49 | 0.77 | 0.05 |
| S10-3-G6 | 3.96 | 0.54 | 2.59 | 0.39 |
| S10-4-C2 | 3.76 | 0.4 | 52.57 | 19.74 |
| S10-6-G4 | 3.78 | 0.72 | 1.30 | 0.06 |
| S10-2-E12 | 3.76 | 0.95 | 1.79 | 0.03 |
| S10-4-C1 | 3.1 | 0.39 | 4.59 | 0.51 |
| S10-6-H3 | 3.27 | 0.6 | 2.01 | 0.09 |
| S10-8-F9 | 3.27 | 0.66 | 2.17 | 0.43 |
| S10-4-F7 | 3.88 | 1.42 | 1.51 | 0.19 |
| S10-1-H7 | 2.54 | 0.16 | 2.14 | 0.24 |

### Microarray Analysis of Spatially Restricted Embryonic Genes

In order to identify genes that are differentially expressed in different regions of early embryos (i.e., genes having “spatially restricted” expression), cells were isolated from the dorsal and ventral marginal zones of an early gastrula stage Xenopus embryo. Cells derived from the ventral marginal zone of vertebrate embryos are the progenitors of mesodermal derivative cells of the developing organism, whereas cells in the dorsal zone, known as “the organizer”, are a source of signals responsible for the induction and patterning of the nervous system. Thus, genes that are differentially expressed in these critical regions of early vertebrate embryo formation are useful, e.g., as markers of these different cell types as well as for the diagnosis and treatment of disorders associated with abnormal embryonic development.

15 μg of total cellular RNA was extracted from the two cell types, differentially labeled and hybridized to the microarrays, as described in Example 2 above. Because total RNA rather than polyA⁺ RNA was used, these experiments are also useful for assessing the feasability of using microarrays when the amount of tissue is limited.

As expected, and in contrast with the results presented in Experiment 3, above, significantly fewer clones were identified that are differentially expressed between the two cell types. Nevertheless, a number of genes did show at least a two-fold difference in expression between the two samples. These included genes such as goosecoid that have been previously shown to be differentially expressed in the dorsal marginal zone. By contrast, other genes represented on the microarray, such as follistatin were not consistently identified as being spatially localized. This result may be due to factors such as low levels of expression, the use of total cellular RNA rather than polyA⁺ RNA and the nature of the genes arrayed.

Genes that showed at least a two-fold difference between the two cell types and a significant hybridization intensity in at least one cell type were selected for RT-PCR and in situ analysis. The RT-PCR analysis of these genes was performed according to the methods described above in Example 4, and the results of this analysis are shown in Table 3. In particular, the table indicates, for each clone that was analyzed by RT-PCR, the average ratio of its expression in the dorsal vs. ventral marginal zone.

30-40% of the genes assayed using RT-PCR changed expression in a direction (i.e. either up- or down-regulated) in a manner that was consistent with the changes observed for those genes on expression arrays. However, the magnitude of the change in expression observed by RT-PCR was different for many genes than the change observed on expression arrays. Further, many of the genes exhibited only very small changes in expression when analyzed using RT-PCR. These differences may have been due to experimental variation in isolating the dorsal and ventral embryonic cells in the two experiments. In general, the data observed by microarray analysis indicates that the genes are more highly regulated than does the RT-PCR data for those genes.

Because the clones selected for RT-PCR analysis in these experiments were detected at much lower levels than are detected at much lower levels than the genes analyzed in Example 4, above, and therefore have a lower signal intensity on microarrays, background noise is a greater factor in analyzing the data. Thus, analysis of total cellular mRNA using the microarrays of this invention are useful for identifying candidate genes whose expression is spatially restricted in early vertebrate embryos. Such candidate genes can then be confirmed, e.g., using more sensitive methods such as the RT-PCR techniques described here, by hybridizing polyA⁺ RNA samples from cells to microarrays, or by using microarrays with more specific and sensitive probes for these candidate genes.

TABLE 3

| | MICROARRAY | RT-PCR |
|---|---|---|
| Clone | Avg. Ratio | Avg. Ratio |
| S10-6-D8 | 2.3 | 1.4 |
| S10-3-C9 | 2.3 | 0.83 |
| S10-3-F3 | 2.4 | 1.0 |
| S10-1-F2 | 2.5 | 2.6 |
| S10-5-H12 | 2.5 | 1.0 |
| S10-8-F7 | 2.7 | 1.13 |
| S10-3-B4 | 3.0 | 1.4 |
| S10-4-H10 | 3.0 | 1.1 |
| S10-2-C6 | 3.2 | 0.87 |
| S10-4-C4 | 3.4 | 1.0 |
| S10-1-A12 | 9.5 | -failed- |
| S10-8-B8 | 0.14 | 9.22 |
| S10-5-C4 | 0.45 | 1.0 |
| S10-2-A1 | 0.47 | 0.89 |

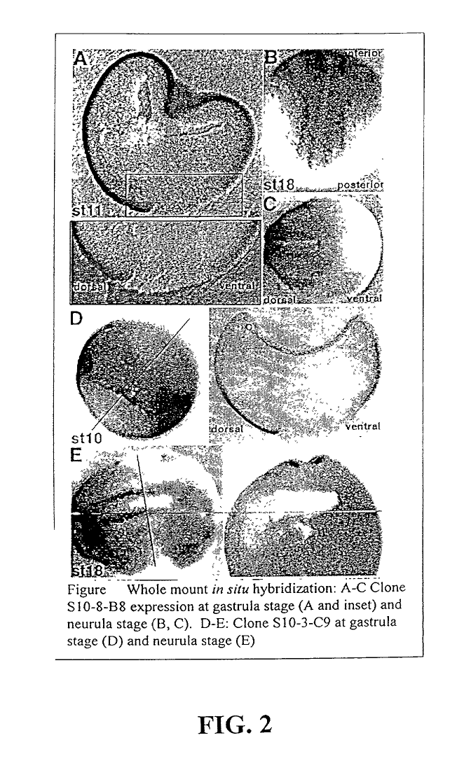

In situ hybridization experiments were also performed to confirm the differential spatial expression of these genes in gastrula stage embryos. Of the twelve clones that were examined by in situ hybridization, three were observed to be differentially expressed during gastrula stages. At later stages, 10 genes were observed to have localized expression patterns. Examples of the differential expression observed for two genes (S10-8-B8 and S10-3-C9) by in situ hybridization are shown in FIG. 2.

### REFERENCES CITED

Numerous references, including patents, patent applications and various publications, are cited and discussed in the description of this invention. The citation and/or discussion of such references is provided merely to clarify the description of the present invention and is not an admission that any such reference is “prior art” to the invention described herein. All references cited and discussed in this specification are incorporated herein by reference in their entirety and to the same extent as if each reference was individually incorporated by reference.

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

What is claimed is:

1. A nucleic acid array, wherein each coordinate of the array contains a single nucleic acid species, which nucleic acid species has a sequence of a Xenopus embryonic gene product set forth in Appendix 1, or the complement thereof, or a hybridizable fragment thereof consisting of not less than 20 contiguous nucleotides from the sequence.

2. The nucleic array of claim 1 comprising all of the sequences from Appendix 1.

3. The nucleic acid array of claim 1 wherein the nucleic acids are cDNAs.

4. The nucleic acid array of claim 1 wherein the nucleic acids are oligonucleotides.

5. The nucleic acid array of claim 1, wherein the array is supported on a solid support selected from the group consisting of a glass slide and a silicon chip.

6. An isolated nucleic acid comprising a sequence corresponding to or complementary to a sequence of not less than 20 contiguous nucleotides of any one of the sequences of Appendix 1.

7. The nucleic acid of claim 6 wherein the sequence consists of the sequence of Appendix 1, or the complement thereof.

8. The nucleic acid of claim 6 wherein the sequence lacks any homology to a known sequence as set forth in the list in Appendix 1.

9. Method for detecting differential expression of embryonic genes, which method comprises:
(a) contacting a nucleic acid array comprising one or more genes expressed in embryonic cells but not in mature cells with a sample nucleic acid preparation and a control nucleic acid preparation, wherein the sample nucleic acid preparation and control nucleic acid preparation contain nucleic acids expressed by sample cells and control cells, respectively, and
(b) detecting differential hybridization of nucleic acids from sample cells relative to control cells to nucleic acids in the array.

10. The method according to claim 9 wherein the sample nucleic acids are mRNAs.

11. The method according to claim 9, wherein the sample nucleic acids are cDNAs produced by reverse transcriptase-polymerase chain reaction (RT-PCR).

12. The method according to claim 11, wherein the sample nucleic acid preparation and the control nucleic acid preparation are each labeled with different labels.

13. The method according to claim 12, wherein the sample nucleic acids are labeled with fluorescent tags.

14. The method according to claim 9, wherein the array is supported on a solid support selected from the group consisting of a glass slide and a silicon chip.

15. The method according to claim 9, wherein the sample cells are at a different developmental point during embryogenesis relative to the control cells.

16. The method according to claim 9, wherein the sample cells are located in a different region of an embryo compared to the control cells.

17. The method according to claim 9, wherein the sample cells are contacted with an external stimulus and the control cells are contacted with a sham stimulus or no stimulus.

18. The method according to claim 17, wherein the cells are contacted with a gene encoding a known gene product.

19. The method according to claim 17, wherein the cells are contacted with a gene encoding an unknown gene product.

20. The method according to claim 17, wherein the sample cells are contacted with a drug.

21. The method according to claim 17, wherein the sample cells are contacted with an environmental toxin.

22. The method according to claim 17, wherein the sample cells are irradiated.

23. The method according to claim 9, wherein the nucleic acid array contains one or more sequences from Appendix 1.

24. Method for detecting defects in development, which method comprises contacting nucleic acids from test cells undergoing development with a nucleic acid array of gene products known to play a fundamental role in the development process, and detecting a difference in expression of a fundamental gene in the sample cells relative to a standard.

25. The method according to claim 24, wherein the standard is a standard derived from expression in a normal cell.

26. The method according to claim 24, wherein the nucleic acid array comprises one or more sequences as set forth in Appendix 1, or the complement thereof, or a hybridizable fragment thereof.

27. The method according to claim 24, wherein a difference in gene expression in test cells relative to normal cells is indicative of a developmental defect.
